(19) United States
(12) Patent Application Publication
(10) Pub. No.: US 2026/0263222 A1
(43) Pub. Date: Sep. 10, 2026

(54) AUGMENTS FOR OSTEO IMPLANTS

(71) Applicant: MERIL HEALTHCARE PVT. LTD, Vapi (IN)

(72) Inventors: VIJAY C. BOSE, Chennai (IN); DARSHAN A. LAD, Bilimora (IN); JIMESH B. GANDHI, Bilimora (IN); PRATIK M. PANCHAL, Vapi (IN); LATHEESH KV, Kannur (IN)

(73) Assignee: MERIL HEALTHCARE PVT LTD, Vapi, GJ (IN)

(21) Appl. No.: 19/491,345

(22) PCT Filed: Jun. 7, 2024
(86) PCT No.: PCT/IN2024/050701

§ 371 Date: Dec. 8, 2025

(30) Foreign Application Priority Data
Jun. 7, 2023 (IN) 202321039119

(51) Int. Cl. A61F 2/30

(52) U.S. Cl. CPC A61F 2/30734; A61F 2002/30168; A61F 2002/30324; A61F 2002/30433; A61F 2002/30736; A61F 2002/30774; A61F 2002/30985; A61F 2310/00023

(57) ABSTRACT

The present disclosure relates to an augment for an implant. The augment includes an elongated member and a lateral member. The elongated member is placed in a gap between a bone and the implant. The elongated member includes a curvature corresponding the curvature of the implant. The lateral member is coupled to the elongated member to be placed on top of the implant. The lateral member is provided at a predefined angle with the elongated member.

### FIELD OF INVENTION

The present disclosure relates to an augment for an osteo implant. More specifically, the present disclosure discloses an augment for an osteo implant for increasing host bone contact.

### BACKGROUND OF INVENTION

People suffering from deteriorating hip joints undergo hip replacement surgery. In the hip replacement surgery, the hip joint is completely or partially replaced by a synthetic implant.

The hip joint includes a femur bone (thigh bone) coupled to a pelvic bone via a ball and socket joint. In the hip replacement surgery, to reinforce the hip joint, the femur bone is provided with a stem prosthesis (or implant).

A conventional stem prosthesis includes an axially extending stem portion and a neck portion that is disposed at an angle with respect to the stem portion. The stem prosthesis is inserted within the cavity of the femur bone. The neck portion indirectly connects the stem portion to the pelvic bone.

Conventionally available stem prostheses are one of a monoblock type or a modular type. As the name suggests, the monoblock stem prosthesis is formed by an integral structure of the neck portion and the stem portion. Monoblock stem prosthesis are easy to insert and have no risk of breakage. However, monoblock stem prosthesis suffer from higher incidents of subsidence and inability to restore soft tissue tension.

In contrast, the modular stem prosthesis has a removably coupled neck portion to the stem portion. The modular stem prosthesis is relatively more resistant to subsidence and provides better ability to achieve soft tissue balance. However, they are more cumbersome to insert within the bone and are prone to break at the point of coupling of the stem portion and the neck portion especially, if the proximal portion of the stem prosthesis is left devoid of bone support.

In about 30% of the cases, it is observed that after inserting the stem prosthesis, there exists an undesirable gap between a proximal portion of the stem and the femur bone such that the proximal portion of the stem prosthesis is left unsupported. Such gaps, if left unnoticed, are prone to implant failure and/or bone (femur) fracture.

Conventionally, a surgeon may be out of options to remedy the undesirable gap between the implant and the bone or may at best use some cement to fill the same which is not an adequate solution.

Therefore, there arises a requirement for a system/device that addresses the aforesaid drawbacks.

### SUMMARY OF INVENTION

Particular embodiments of the present disclosure are described herein below with reference to the accompanying drawings; however, it is to be understood that the disclosed embodiments are mere examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present disclosure relates to an augment for an implant. The augment includes an elongated portion and a lateral member. The elongated portion and the lateral member form an integral structure. The elongated portion is placed in a cavity defined between a bone and the implant and includes a curvature corresponding to the curvature of the implant. The lateral member and the lateral member of the augment form a predefined angle. The lateral member is placed on top of the implant and coupled to the elongated member.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts a cross-section of a femur bone 1 with an implant 10 and augment 100 in accordance with an embodiment of the present disclosure.

FIG. 2 depicts the augment 100 for the implant 10 in accordance with an embodiment of the present disclosure.

FIG. 3 depicts the implant 10 in accordance with an embodiment of the present disclosure.

FIG. 4 depicts a longitudinal cross-section of a femur bone 1 with the implant 10 and augment 100 in accordance with an embodiment of the present disclosure.

### DETAILED DESCRIPTION OF DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can 5 encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In the context of the present disclosure, a gap refers to the space between a bone and an implant, once the implant is inserted. The term cavity refers to a reshaped or redefined gap in which an augment is inserted. Typically, the dimensions of the cavity correspond to the dimensions of the augment.

The present disclosure discloses an augment for an osteo implant (or implant). The augment is snugly placed within a cavity between an implant and an inner surface of the bone. The augment thus minimizes the gap between an implant and an inner surface of the bone and maintains maximum implant-bone contact. Further, the risk of subsidence is substantially reduced.

The augment of the present invention is designed to be installed in the femur bone of a patient during the surgery. However, it is envisaged that the augment can be used along with a humeral implant for shoulder surgery with some suitable adaptations. The configuration of the augment in such embodiments may be substantially as described below, although the overall size and shape of the augment would be configured to allow it to be installed in a humerus instead of a femur. It is also envisaged that at least some embodiments of this invention may instead apply in the context of a (distal) femoral implant or a tibial implant for knee surgery. Again, the configuration of the implant in such embodiments may be substantially as described below, although the overall size and shape of the augment would be configured to allow it to be installed in a tibia or distal femur bone instead of a proximal femur.

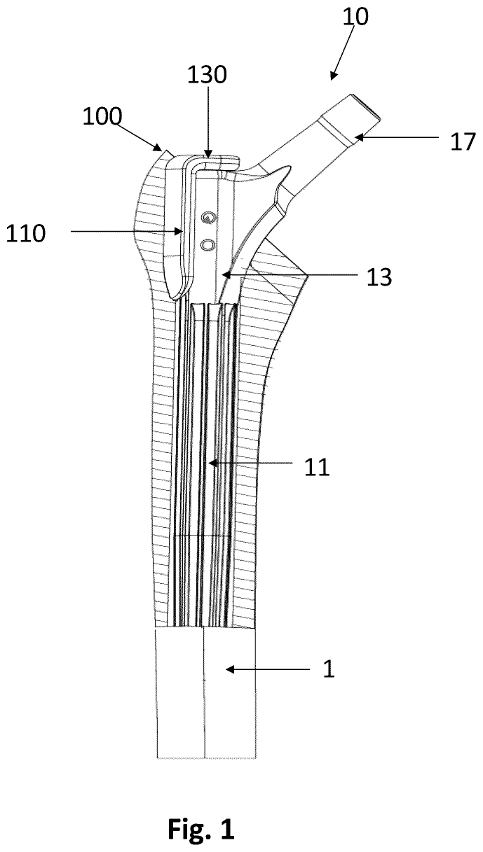

Now referring to figures, FIG. 1 depicts a cross-section of a femur bone 1 with the implant 10 and an augment 100. The augment 100 minimizes the gap between the implant 10 and the femur bone 1 thereby minimizing implant failure and/or femur bone fracture. The augment 100 is mounted on the implant 10 in the femur bone 1. The augment 100 may extend at least partially within the femur bone 1 along the surface of the implant 10.

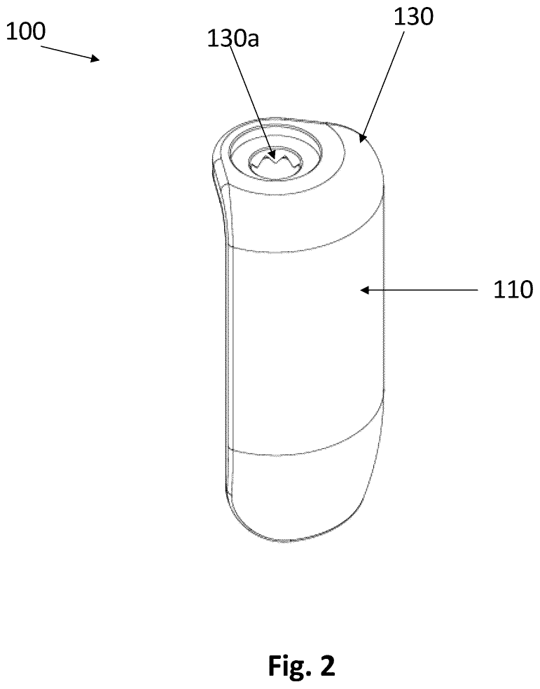

The augment 100 includes an elongated member 110 and a lateral member 130, details of which are described in FIG. 2. The elongated member 110 includes two opposite faces. Once mounted on the implant 10, the elongated member 110 of the augment 100 is disposed axially along the length of the body 13 of the implant 10. One face of the elongated member 110 is in contact with the implant 10 and the other face of the elongated member 110 is in contact with the inner surface of the femur bone 1. The lateral member 130 is configured to be coupled on top of a shoulder 15 of the implant 10 as described below for proper fixation of the augment 100 to the implant 10.

The augment 100 thus, provides support to the implant 10 by filling the gap between the body 13 and the inner surface of the femur bone 1.

FIG. 2 depicts the augment 100 for the implant 10 utilized for reducing the gap between the implant 10 and an inner surface of the femur bone 1. Essentially, the augment 100 is coupled to the shoulder 15 of the implant 10 to reduce/eliminate the gap between the implant 10 and an inner surface of the femur bone 1.

The augment 100 is made of a porous material including but not limited to titanium, cobalt chromium, and other metals or alloys thereof. The augment 100 is made either by 3D printing or by machining. In an exemplary embodiment, the augment 100 is made by 3D printing using a titanium alloy (Ti-6Al-4V-ELI). The augment 100 helps to support the body 13 (and the shoulder 15) of the implant 10 thereby reducing the risk of subsidence. Further, the augment 100 helps in enhanced osteointegration of the implant 10 which provides further resistance against subsidence. The augment 100 also helps to utilize the trochanteric fragment with the body 13 of the implant 10 if the trochanteric bone is of poor quality or fractured.

As stated above, the augment 100 includes the elongated member 110 and the lateral member 130. In an exemplary embodiment, the elongated member 110 and the lateral member 130 form an integral structure.

The elongated member 110 includes a plate-like body having optionally, a curvature. In an exemplary embodiment, the radius of the curvature of the elongated member 110 corresponds to the radius of the body 13 on which the augment 100 is attached, for example, the body of a stem. The edges of the plate-like body may be smooth, chamfered, rounded etc. The distal portion of the elongated member 110 may be tapered or rounded. Alternately, the distal portion of the elongated member 110 may be straight. The length of the elongated member 110 is at least 5 mm.

The elongated member 110 and the lateral member 130 may have a pre-defined thickness. In an exemplary embodiment, the thickness of the elongated member 110 and the lateral member 130 is same. Alternatively, the thickness of the elongated member 110 and the lateral member 130 may be different. In an exemplary embodiment, the thickness (say, at least 1mm) of the elongated member 110 corresponds to the width of a cavity defined between the body 13 of the implant 10 and an inner surface of the femur bone 1, post reaming a gap between the two. The thickness of the gap present between the body 13 and the inner surface of the femur bone 1 varies based on the anatomy of the femur bone 1. The minimum thickness of the elongated member 110 is 1 mm. The minimum thickness of the lateral member 130 is 1 mm.

In an embodiment, the thickness of the elongated member 110 and the lateral member 130 varies along their respective surfaces. For example, the thickness of the elongated member 110 and the lateral member 130 towards the edges may be less than the thickness around their respective central portions. Alternately, the thickness of the elongated member 110 and the lateral member 130 may be varied as per the support required to be provided to the implant 10 and all such variations are within the scope of the present disclosure.

The shape and surface of the augment 100 is critical for enhanced osteointegration of the implant 10. In an exemplary embodiment, the augment 100 includes a textured or a rough or a latticed surface to promote bone ingrowth or osteointegration.

The lateral member 130 may define a predefined angle with the elongated member 110. The predefined angle between the elongated member 110 and the lateral member 130 may correspond to the anatomy of the femur bone 1. In an exemplary embodiment, the predefined angle between the elongated member 110 and the lateral member 130 is 90° resembling an inverted L.

The lateral member 130 may be provided with one or more holes 130a. The number of holes 130a corresponds to the number of recesses 15a of the shoulder 15. The augment 100 is secured to the shoulder 15 of the implant 10 by using a fastening mechanism including but not limited to snap fit, taper locking, fastener, etc. The fastening mechanism passes through the hole 130a of the augment 100 and into the recess 15a of the implant 10 etc. thereby, securing the two. In an exemplary embodiment, the fastener 130b is used to fix the augment 100 in the desired orientation with respect to the implant 10. Other functionally equivalent means is within the scope of the teachings of the present disclosure.

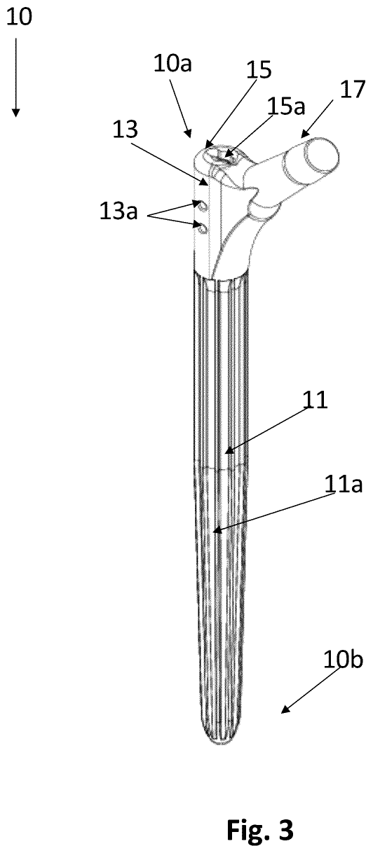

Before describing the assembly of the augment 100 and the implant 10, an exemplary implant is described in FIG. 3. It is to be noted that the augment 100 can be used with any implant including the exemplary embodiment of FIG. 3. The implant 10 is configured to be inserted within a cavity of a bone to reinforce the bone (and/or an adjacent joint). In an exemplary embodiment, the implant 10 is inserted within a cavity defined in the femur bone 1 to reinforce a hip joint.

In an exemplary embodiment, the implant 10 is inserted within the cavity of the femur bone 1 by using an implant applicator as described in Indian patent application number 202321039103.

The implant 10 has a proximal end 10a and a distal end 10b. Further, the implant 10 includes a stem 11, a body 13, a shoulder 15 and a neck 17. The stem 11 is disposed towards the distal end 10b of the implant 10 such that the distal tip of the stem 11 defines the distal end 10b of the implant 10.

The stem 11 may be tubular, conical, cylindrical or any other suitable shape. As depicted, the stem 11 is cylindrical in shape. In an exemplary embodiment, as shown in FIG. 1, the stem 11 is at least partially tapered towards the distal end 10b. That is, the middle section of the stem 11 has a larger diameter than the distal section. The distal end of the stem 11 may be pointed. Alternately, the distal end of the stem 11 may be of any other shape including, but not limited to curved, round, bullet, etc. for easy insertion of the implant 10 into the bone. The tapered structure of the stem 11 increases the utility of the implant 10 as it facilitates a more efficient implant insertion process, minimizing operative time and surgeon effort.

Additionally or optionally, the stem 11 is provided with a plurality of ribs 11a on an outer surface of the stem 11. The ribs 11a may be distributed circumferentially on the outer surface of the stem 11. The ribs 11a may extend at least partially on the outer surface of the stem 11 along the axis of the stem 11 (or in the longitudinal direction). In an embodiment, the ribs 11a extend longitudinally on the outer surface of the stem 11, from the distal to proximal end.

In an embodiment, the ribs 11a have a textured surface facilitating the implant 10 to have a friction fit with the surrounding bone after the implant 10 is inserted within the cavity of the bone. The ribs 11a can include various patterns including but not limited to linear, zig-zag, wavy, etc. In an exemplary embodiment, the ribs 11a have a linear pattern on the surface of the stem 11. The ribs 11a provide rotational resistance to the implant 10 within the cavity of the bone and increase the total host bone contact surface area.

The body 13 is disposed towards the proximal end 10a of the stem 11. In an exemplary embodiment, the body 13 forms an integral structure with the stem 11. Alternately, the body 13 may be removably coupled to the stem 11.

Additionally or optionally, the body 13 is provided with one or more holes 13a. In an exemplary embodiment, the holes 13a in the middle region of the body 13 enable a user to attach wire(s) to a trochanteric fragment (not shown). This trochanteric fragment functions as a synthetic replacement for the natural trochanteric bone, an anatomical structure located on the upper portion of the femur. The trochanteric fragment is utilized when the trochanteric bone is of poor quality or fractured.

The body 13 includes an outer surface which stays in direct contact with the femur bone 1 (or bone) once implanted in the femur bone 1. The outer surface of the body 13 may be provided with at least one layer of coating. In an exemplary embodiment, the outer surface of the body 13 is provided with one layer of coating including but not limited to hydroxyapatite (HA), titanium coating, etc. The coating has chemical similarity to the minerals of a natural bone and thus, helps in enhanced bone-implant integration of the implant 10 and femur bone 1, after the implant 10 is inserted within the cavity of the femur bone 1. The coating further promotes regrowth of the bone tissues resulting in increased rate of osteointegration.

The shoulder 15 is disposed at the proximal end 10a of the implant 10. The shoulder 15 is provided with at least one recess 15a. The recess 15a can be circular, semi-circular, oval, or any other desirable shape. Further, the wall of the recess 15a may be threaded, semi-threaded or unthreaded. The recess 15a extends at least partially along the length of the body 13. Further, the recess 15a may be aligned with the center of the shoulder 15 or may be offset from the center by a predefined distance.

The recess 15a helps a user to couple the augment 100 to the implant 10 using a coupling mechanism including but not limited to a fastener, a rivet etc. In an exemplary embodiment, the recess 15a couples the augment 100 to the shoulder 15 of the implant 10. The coupling mechanism includes without limitation a fastener 130b (as shown in FIG. 2), press-fit, tapered fit, etc. The fastener 130b can be made of any biocompatible material including but not limited to titanium, cobalt chromium, and other metals or alloys thereof. In an exemplary embodiment, the fastener 130b is made of titanium.

The neck 17 extends away from the body 13 of the implant 10. The neck 17 defines a pre-defined angle with the axis of the stem 11. The said angle is based upon the orientation of the femur bone with an adjacent pelvic bone. The neck 17 indirectly forms a ball and socket joint with the pelvic bone. In an exemplary embodiment, not shown, the neck 17 is provided with a ball that is coupled to a native socket of the pelvic bone or an implant provided on the pelvic bone forming a ball and socket joint replicating the motion of the natural joint.

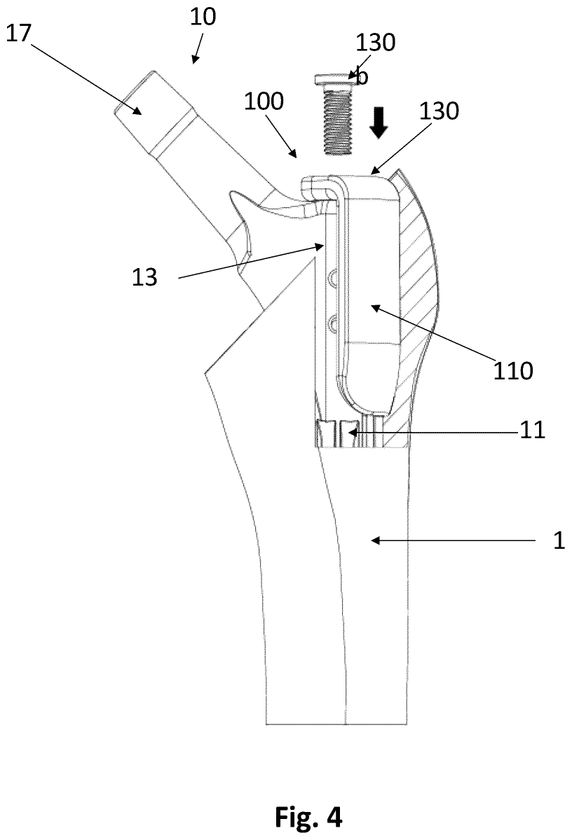

FIG. 4 illustrates an exemplary exploded view of the assembly. First, an implant 10 is inserted in the femur bone 1. For this, initially the stem 11 of the implant 10 is inserted. After proper positioning of the stem 11 of the implant 10 in the bone 1, the gap between the implant 10 and the bone cavity is analysed. The gap is reamed to obtain a cavity of suitable thickness. Once the cavity is formed, an augment of corresponding thickness is chosen.

Thereafter, the augment 100 is mounted on the implant 10. Specifically, the elongated member 110 of the augment 100 is inserted in the cavity between the implant and the bone. The augment 100 is pushed till the lateral member 130 of the augment 100 is placed on the shoulder 15 of the implant 10. The two are then coupled using the fastener 130b.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. An augment (100) for an implant (10), comprising:
an elongated member (110) to be placed in a cavity defined between a bone 1 and the implant (10); and
a lateral member (130) coupled to the elongated member (110), to be placed on top of the implant (10);
wherein the lateral member (130) is provided at a predefined angle with the elongated member (110);
wherein the elongated member (110) includes a curvature corresponding to the curvature of the implant (10).

2. The augment (100) as claimed in claim 1, wherein edges of at least one of the elongated member (110) and the lateral member (130) are smooth or chamfered.

3. The augment (100) as claimed in claim 1, wherein the elongated member (110) is of varying thickness.

4. The augment (100) as claimed in claim 1, wherein the lateral member (130) includes a hole 130a to allow passage of a coupling mechanism.

5. The augment (100) as claimed in claim 4, wherein the hole 130a is one of a threaded, semi-threaded or unthreaded hole.

6. The augment (100) as claimed in claim 4, wherein the coupling mechanism includes a fastener 130b.

7. The augment (100) as claimed in claim 1, wherein the elongated member (110) and the lateral member (130) form an integral component.

8. The augment (100) as claimed in claim 1, wherein the augment (100) is 3D printed or machined.

9. The augment (100) as claimed in claim 1, wherein the augment (100) is made of a titanium alloy.

10. The augment (100) as claimed in claim 9, wherein the augment (100) includes a textured or rough surface.

11. The augment (100) as claimed in claim 1, wherein the predefined angle between the elongated member (110) and the lateral member (130) is 90° resembling an inverted L.
